(19) United States
(12) Patent Application Publication
(10) Pub. No.: US 2024/0307071 A1
(43) Pub. Date: Sep. 19, 2024

(54) BONE CUTTING TOOL FOR REDUCTION OF PLANAR DEFORMITIES OF VARIABLE MAGNITUDE

(71) Applicants: Mack Jay GROVES, IV, Mandeville, LA (US); Robert Brian WEINSTEIN, Atlanta, GA (US)

(72) Inventors: Mack Jay GROVES, IV, Mandeville, LA (US); Robert Brian WEINSTEIN, Atlanta, GA (US)

(21) Appl. No.: 18/604,938

(22) Filed: Mar. 14, 2024

Related U.S. Application Data
(60) Provisional application No. 63/576,913, filed on Mar. 15, 2023.

(51) Int. Cl. A61B 17/15

(52) U.S. Cl. CPC A61B 17/152

(57) ABSTRACT

A handle and a cutting guide each removably mount to a wedge so that they are reconfigurable between a first/positioning configuration and a second/cutting configuration. In the first configuration, the handle is mounted to the wedge to form a joint-repositioning orthopedic surgery tool for use in correcting orthopedic deformities. And in the second configuration, the cutting guide is mounted to the wedge to form a saw-guide orthopedic surgery tool for use in correcting orthopedic deformities. Methods of correcting orthopedic deformities include positioning the wedge of the joint-repositioning tool between two bones of a joint to reposition at least one of the bones, and using the cutting guide of the saw-guide tool to saw off at least one bone end portion, to form bone end edges that are substantially parallel so the bones are now substantially aligned.

### CROSS-REFERENCE TO RELATED APPLICATION

This application is claims priority to U.S. Provisional Patent Application Ser. No. 63/576,913 filed Mar. 15, 2023, which is incorporated herein by reference.

### TECHNICAL FIELD

The present invention relates generally to the field of orthopedic surgery and, in particular, surgical devices and methods for reducing deformities in joints.

### BACKGROUND

It is common in the field of orthopedic surgery to perform operations involving bones where the procedural demands require reduction of a deformity existing at or near a joint. Such deformities can cause pain secondary to mechanical axis deviation or downstream effects such as bone prominences or instability. When a surgeon is faced with this situation, it is often the desire to reduce the deformity to anatomic standard and maintain this corrected alignment. Historically this has been performed using manual reduction (simply “squeezing” the area into a corrected position) or bone clamps (mechanically correcting using leverage). After this reduction, bone cuts can be made that can alter the axis of deformity, allow for placement of an implanted device to maintain this new angle, or remove cartilage from adjacent sides of a joint at an angle that when fixated with screws, plates, or staples will maintain the corrected state.

More recently, combinations of clamps and jigs have been employed to reduce these angular deformities based on prescribed values. However, the process of deformity reduction is the same with these methods, that is, exposure of the affected joint, loosening of soft tissue attachments to mobilize the joint, reduction of the deviated bone to anatomic parameters, and then fixing the joint in the corrected position.

To determine if a joint is “deviated,” or out of anatomic standard values, a surgeon would rely on radiographic measurements to assess the level of deviation both in apex and magnitude. This way the surgeon can then know where to focus and the degree of correction required to achieve the anatomic standard. If the surgeon has this information preoperatively, a cutting guide properly placed can assist in making bone cuts that will achieve angle reduction. There are several devices that have recently come to the market that fulfill this purpose. Using these devices, the surgeon determines and measures the angle of deviation preoperatively, affixes the device in surgery, and the instrumentation is assembled according to the pre-operative value. This may take the form of a cutting block at a specific degree, or rotating a cutting guide to the specific degree. Once the device is attached, the bone cuts are made at the predetermined angle. The device is removed with the bone cut at an angle such that it measures the preoperative value. In cases where bone is removed, this cut angle theoretically matches the preoperative deformity value and consequently the deformity is reduced to anatomic standards.

But there are problems with this method. First, there is variability in radiographic angles that exist based on position of the foot and position of the X-ray tube head that may enhance or subtract from the true angular deviation calculated prior to surgery. If this angle is incorrectly obtained or measured, the settings on the surgical device will carry this incorrect measurement into the bone cuts leading to under- or over-correction of the deformity. Second, the devices can be cumbersome, often with multiple linked attachments that require a great deal of time and dexterity to assemble. Third, these devices often affix to adjacent non-operative bones for stability, introducing second-site comorbidity. Fourth, these devices use an indirect method of joint deformity reduction. Instead of applying a realigning device at the center of the actual joint deformity, an extra-articular jig is applied at a distance away from the joint. Inherently, this indirect joint manipulation approach is less precise and may result in over or under-correction of the deformity. Fifth, the devices are often linked to corresponding fixation methods, making it unavoidable for the surgeon to use alternative fixation methods. Fifth, the devices are extremely expensive due to their complex mechanical nature. Sixth, in devices using pre-angled bone cutting blocks, the apparatus cannot accommodate for an infinite degree of deformity, but rather the specified values based on the design of the blocks. Once cuts are made, there is no titration of angles, and only after the bone has been removed can the surgeon assess if the cut angles were appropriate for full reduction but not over-reduction of the deformity.

Calculation of a true angle of deviation is critical for proper reduction to anatomic standards. Recently computed tomography (CT) studies have overcome some of the variability to traditional plain-film methods of calculation. However, this added cost and radiation exposure is very often unnecessary, for example in cases of bunion correction. This relatively benign condition is consequently magnified in complexity and expense to the patient and health care system when CT evaluation is used. One additional consideration to this angular measurement scenario in the real-world application of weight-bearing measurements preoperatively correlating to those appreciated while a patient is laying down on an operating table in a supine, non-weight bearing attitude. Regardless of plain-film or CT acquired measurements, both obtained in weight-bearing attitudes, the surgeon does not operate on weight-bearing patients. Therefore, the preoperative calculation, regardless how obtained, is very often inconsistent with deformity measured in the operating room. Devices with multiple attachments, settings, jigs, and blocks require surgeon education and practice, and manufacturer representation during the surgery, to ensure correct application occurs. There is a high degree of probability that as device complexity increases, so do error rates. Additionally, when there are multiple linkage points, there are multiple points where loosening or deviations can occur during or after apparatus placement. This is especially true when subjected to manipulation of a body part or with the rapid vibration of a bone saw. When additional bones are used for anchor points, their violation introduces potential for neurovascular or tendon damage, bone infection, and stress fractures. It is always a desire to avoid additional bone or soft tissue injury during a procedure, making this a detrimental requirement in the assembly of any apparatus.

There are several principles involved in reducing a deformity at a joint by osteotomy. First, angular deviation from standards must be able to be determined and consequently corrected on the operative field. Second, the instrumentation to create these bone cuts must be sufficiently robust to allow the surgeon to assess in real time whether or not the deformity will be reduced. Third, the instrumentation must not interfere with the surgeon's approach to the joint. Fourth, the angle reduction must be able to be maintained after the corrective bone cuts are made such that the deformity stays reduced.

Thus, there exists a need for a device that can overcome the deficiencies of the prior art.

### SUMMARY

Generally described, the present invention relates to orthopedic surgical systems and methods for reducing angular deformities in joints. The orthopedic surgical systems include a handle and a cutting guide that each removably mount to a serrated wedge so that they are reconfigurable between a first/positioning configuration and a second/cutting configuration. In the first configuration, the handle is mounted to the wedge to form a joint-repositioning orthopedic surgery tool for use in correcting orthopedic deformities. And in the second configuration, the cutting guide is mounted to the wedge to form a saw-guide orthopedic surgery tool for use in correcting orthopedic deformities. Orthopedic surgical methods of correcting orthopedic deformities include positioning the wedge of the joint-repositioning tool between two bones of a joint to reposition at least one of the bones, and using the cutting guide of the saw-guide tool to saw off at least one bone end portion, thereby forming bone end edges that are substantially parallel so the bones are now substantially aligned.

In various embodiments, the systems and methods can provide the following:
1. An apparatus for facilitating reduction of an angular deformity comprised of a serrated wedge, the wedge being a scalene triangle of a fixed degree;
2. The apparatus above that can be inserted to any distance between two bones, and as a triangular shape will generally cause a change in the angular relationship between two bones at the joint in between;
3. The apparatus above that due to its geometry and wedge effect will cause the angular relationship between adjacent bones to change commensurate with the depth of insertion, including an infinite number of degrees between 0 at entry to the maximum of the most acute angle of the leading tip of the wedge;
4. The apparatus above having serrations on bone contact faces arranged to prevent backing out or extrusion of the wedge when inserted;
5. the apparatus above that may or may not be marked to indicate angle correction points along its length;
6. The apparatus above having a connection point for attachment to a handle temporarily to aid in placement, with the ability to be struck by a mallet or heavy object;
7. The apparatus above having a connection point for attachment to a cutting guide/block;
8. The apparatus above, when connected to a cutting guide/block, that can aid in parallel cuts in two adjacent bones separated by the serrated wedge;
9. The apparatus above that can optionally affix to a cutting guide/block of prescribed angled slots instead of parallel slots;
10. The apparatus above, consisting of 3 key components, that can be assembled readily on the surgical field, can be utilized on either right or left side, and that is fixation agnostic.

These and other aspects, features, and advantages of the invention will be understood with reference to the drawing figures and detailed description herein and will be realized by means of the various elements and combinations particularly pointed out in the appended claims. It is to be understood that both the foregoing general description and the following brief description of the drawings and detailed description of example embodiments are explanatory of example embodiments of the invention, and are not restrictive of the invention, as claimed.

### BRIEF DESCRIPTION OF THE DRAWINGS

FIG. 1 is an exploded top view of an orthopedic surgical device system for reducing angular deformities in joints according to a first example embodiment, showing a handle and a cutting guide that are each removably mountable to a wedge to selectively form a joint-repositioning tool and the saw-guide tool.

FIG. 1A is a top view of the wedge of FIG. 1, with wedge serrations not shown for simplicity.

FIG. 2 is a side view of the wedge of FIG. 1.

FIG. 3 is a perspective view of the wedge of FIG. 1.

FIG. 4 is a proximal end view of the wedge of FIG. 1.

FIG. 5 is another perspective view of the wedge of FIG. 1.

FIG. 6 is a perspective view of the handle of FIG. 1.

FIG. 7 is a distal end view of the handle of FIG. 1.

FIG. 8 is a perspective view of the handle of FIG. 1 from the opposite end as shown in FIG. 6.

FIG. 9 is an exploded top view of the wedge and handle of FIG. 1, showing these components disassembled, with wedge serrations not shown for simplicity.

FIG. 10 shows the wedge and handle of FIG. 9 assembled together in a first system configuration to form the joint-repositioning tool, with wedge serrations not shown for simplicity.

FIG. 11 is a proximal end view of the cutting guide of FIG. 1.

FIG. 12 is a perspective view of the cutting guide of FIG. 1.

FIG. 13 is an exploded perspective view of the wedge and cutting guide of FIG. 1, showing these components disassembled.

FIG. 14 shows the wedge and handle of FIG. 13 partially assembled together in a second system configuration to form the saw-guide tool.

FIG. 15 is a top view of the wedge and handle of FIG. 14, shown fully assembled together to form the saw-guide tool, with wedge serrations not shown for simplicity, and also showing the proximal/head end portion of the handle used in the assembling process.

FIG. 16 a top view of a wedge of an orthopedic surgical device system for reducing angular deformities in joints according to a second example embodiment.

FIG. 17 is a side view of the wedge of FIG. 17.

FIG. 18 is a proximal end view of the wedge of FIG. 17.

FIG. 19 a top view of a handle of the orthopedic surgical device system including the wedge of FIG. 17.

FIG. 20 is a proximal end view of the handle of FIG. 19.

FIG. 21 is a distal end view of the handle of FIG. 19.

FIG. 22 is a cross-sectional view of the handle taken at line 22-22 of FIG. 19.

FIG. 23 is proximal end of a cutting guide of the orthopedic surgical device system including the wedge and handle of FIGS. 17 and 19.

FIG. 24 is a top view of the cutting guide of FIG. 23.

FIG. 25 is a side view of the wedge and handle of FIGS. 17 and 19, shown removably mounted together to form a joint-repositioning tool.

FIG. 26 is a top view of the wedge and cutting guide of FIGS. 17 and 23, shown removably mounted together to form a saw-guide tool.

FIG. 27 is a top view of the wedge and cutting guide of FIG. 26, shown partially assembled together.

FIG. 28 is a proximal end view of the wedge and cutting guide of FIG. 27.

FIG. 29 is a proximal end view of a wedge and cutting guide of an orthopedic surgical device system for reducing angular deformities in joints according to a third example embodiment for which the two adjacent bones need not be aligned with each other before cutting.

FIG. 30 shows the wedge and cutting guide of FIG. 29 in an angularly adjusted orientation.

FIG. 31 is a perspective view of cutting guide of an orthopedic surgical device system for reducing angular deformities in joints according to a fourth example embodiment for which the two adjacent bones need not be aligned with each other before cutting.

FIG. 32 is a top view of the cutting guide of FIG. 31 removably mounted to a wedge of the orthopedic surgical device system to form a saw-guide tool, shown in use in a joint between the end portions of two bones for deformity correction.

FIG. 33 is a side view of the saw-guide tool including the cutting guide and wedge of FIG. 32 in use for deformity correction.

FIG. 34 is a top view of a cutting guide and a truncated wedge of an orthopedic surgical device system for reducing angular deformities in joints according to a fifth example embodiment, showing the cutting guide and truncated wedge removably mounted together to form a saw-guide tool, shown in use in a joint between the end portions of two bones for bi-planar deformity correction.

FIG. 35 is a side view of the saw-guide tool including the cutting guide and wedge of FIG. 34 in use for bi-planar deformity correction.

FIGS. 36A-36H are schematic views of a method of correcting orthopedic deformities according to an example embodiment of the invention.

### DETAILED DESCRIPTION OF EXAMPLE EMBODIMENTS

The present invention may be understood more readily by reference to the following detailed description of example embodiments taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. Any dimensions identified in the specification or drawings are intended to be representative for illustration purposes and not limiting of the invention, unless recited in the claims.

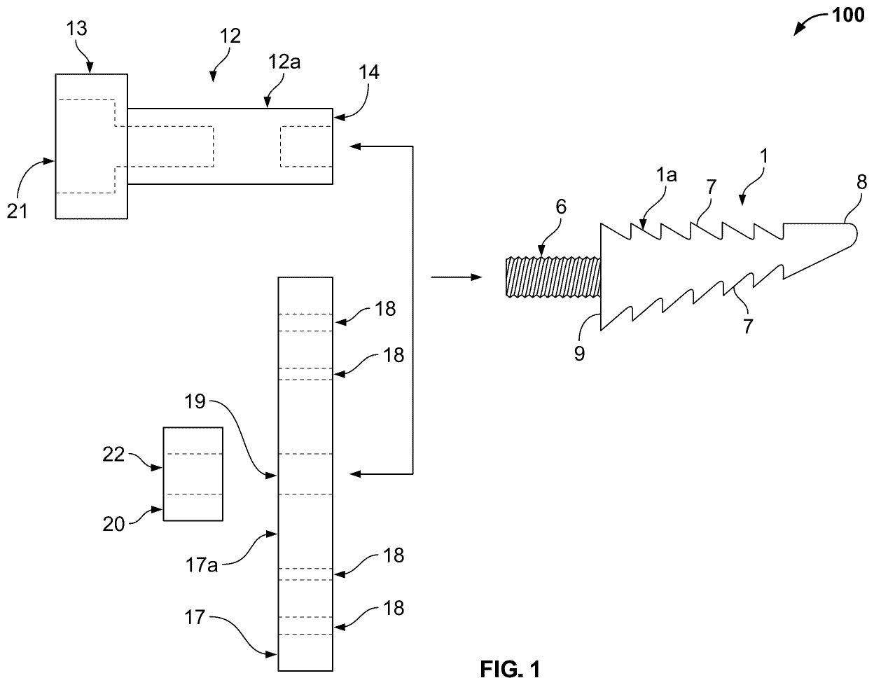

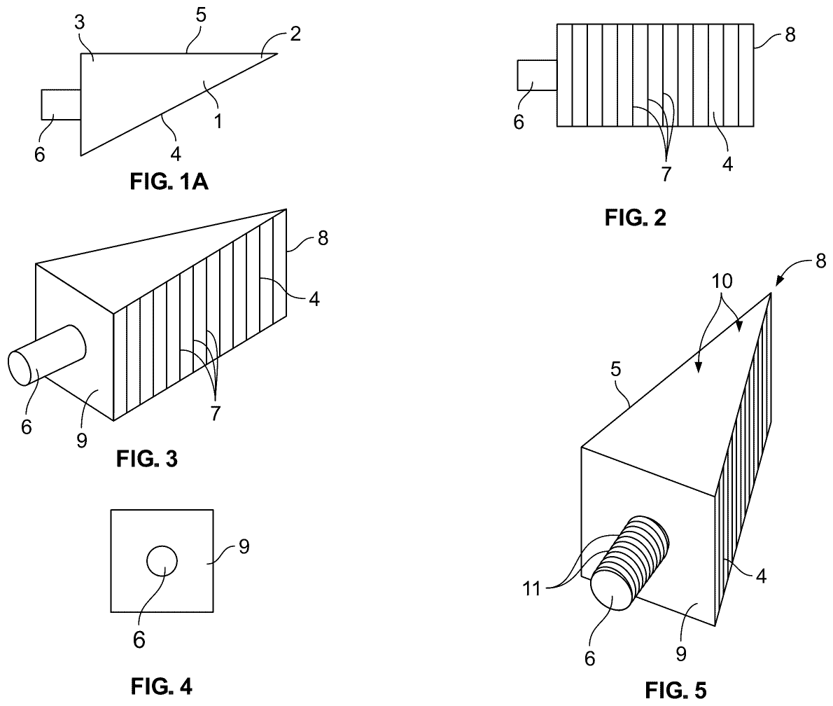

With reference now to the drawing figures, wherein like reference numbers represent corresponding parts throughout the several views, FIGS. 1-15 show an orthopedic surgical device system 100 for reducing angular deformities in joints according to a first example embodiment. The surgical system 100 can be used in orthopedic surgical methods to reduce a deformity existing at a joint level and cut bones on either side to achieve a permanently corrected position. In particular, the surgical system 100 can be used to wedge a joint open to a certain degree in a first configuration, then rearranged into a second configuration to include a cutting guide for specific bone cuts that will ultimately permit fixation in this permanently reduced state.

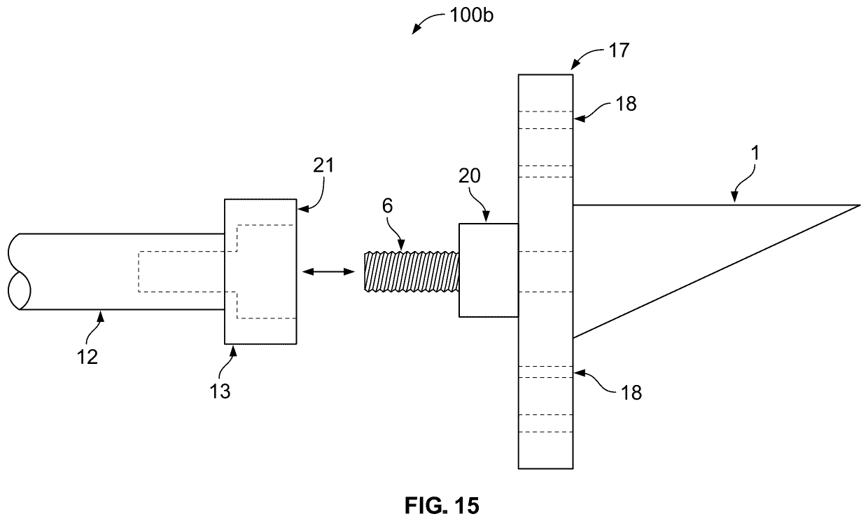

The surgical system 100 includes a wedge 1, a handle 12, and a cutting guide 17, with the handle 12 and the cutting guide 17 each removably mountable to the wedge 1. The surgical system 100 is reconfigurable between the first/positioning configuration with the handle 12 mounted to the wedge 1 to form a joint-repositioning tool 100a (see FIG. 10) and the second/cutting configuration with the cutting guide 17 mounted to the wedge 1 to form a saw-guide tool 100b (see FIG. 15). The wedge 1, the handle 12, and the cutting guide 17 can all be made using conventional materials and fabrication techniques suitable for surgical devices.

Referring to FIGS. 1-5, the wedge 1 includes a tapered block 1a and a wedge coupling part 6. The tapered block 1a is wedge-shaped (i.e., triangular in at least one plane) with a distal (leading/apex) tip 8 opposite a proximal (base) face 9. The wedge block 1a has two opposing faces 4 and 5 that are angled relative to each other, and that intersect at the leading tip 8 and intersect the base face 9 to form the wedge shape. The wedge shape is configured to insert into a joint between bones and directly contact the cartilage therein. The two angled faces 4 and 5 are typically vertically oriented in surgical use, with the two opposing top and bottom faces extending between them. The angled faces 4 and 5 have serrations 7 formed on them in a configuration such that they dig into the joint cartilage and/or bone to secure the wedge 1 when placed into the joint.

Referring specifically to FIG. 1A, in the depicted embodiment, the block 1a is a scalene wedge and the wedge coupling 6 is a threaded male projection (e.g., a bolt or other extension element) on one side. The apex angle is of varied degree, although one other degree is 90. The wedge block 1a has generally a triangular shape an acute apex angle 2, for example between 15 and 30 degrees, with a second angle 3 arranged such that the triangle-shaped block 1a forms a scalene triangle wedge, and with the second angle 3 for example 90 degrees to the triangle base face 9. The angled faces 4 and 5 are configured to insert into a joint between bones and directly contact the cartilage therein. The threaded projection 6 extends from the base face 9 of the wedge block 1a.

Referring specifically to FIG. 2, in the depicted embodiment, the wedge block 1a has a greater length than height, with the serrations 7 along the face 4 arranged in a manner to extend along the entire side to dig into the joint cartilage to secure the wedge 1 when placed in the joint. The leading edge 8 is a tapered point and leads the wedge 1 entry into the joint. The depicted wedge block 1a has generally a length to width ratio of about 3:1, though other embodiments include alternate dimensions and dimensional ratios to achieve the same generally triangular shape and produce the same functionality described herein. The depicted wedge block 1a is at least 20 millimeters in depth and 15 millimeters in height, though in other embodiments it would be functionally similar to have a wedge block 1a of similar geometric shape that performed as a wedge no matter how scaled larger or smaller.

In addition, it will be understood to those skilled in the art that the wedge block can have a triangular shape in a second plane, for example forming a truncated wedge, so that the wedge block would have a triangular/wedge shape in both FIGS. 1 and 2. Thus, in FIG. 2, the top and bottom of the wedge would have different widths, that is, the top and bottom faces of FIG. 2 would be angled with respect to each other, just as the two side faces 4 and 5 of FIG. 1A are angled with respect to each other. Using the wedge of this embodiment, more than one plane of correction can be accomplished in the joint.

Referring specifically to FIG. 3, in the depicted embodiment, the base face 9 is flat and the threaded projection coupling 6 extends centrally from it. Arranged along the angled faces 4 and 5 are the serrations 7 that span substantially the entire length of the faces 4 and 5 on either side of the wedge block 1a. As depicted, the serrations 7 are oriented generally vertically in the faces 4 and 5 and arranged at an angle to resist motion backwards toward the base face 9 once the wedge 1 is inserted into a joint beginning with the leading edge 8. A second effect of these serrations 7 is to increase total surface area of the wedge block 1a to thereby increase the total surface contact with the internal edge of the joint, which increases stability when the wedge 1 is inserted therein.

Referring specifically to FIG. 4, in the depicted embodiment, the base 9 face of the wedge block 1a has the threaded projection coupling 6 positioned in the center of the face 9. The projection 6 can extend from the wedge block 1a in line with the center of gravity of the wedge block 1a to best direct forces (e.g., when hit with a mallet).

This projection coupling 6 allows for removable attachment of optional components/devices that will be further described herein. It can be appreciated that the base face 9 is generally rectangular.

Referring specifically to FIG. 5, in the depicted embodiment, the serrations 7 are arranged along at least the angled face 4. Markings 10 can be formed along the wedge block 1a to indicate depth, angle, distance, or some other measure of the wedge 1 inserted into the joint. For example, the markings 10 can detail the linear or angular dimension from apex 8 to face 9. The markings can be formed along the angled face 4 or 5, one of the two faces extending between then, or one of the edges defined by those faces. The markings 10 can include millimeters, centimeters, degrees, or other parameters, based on the measurement to be indicated. One skilled in the art can understand these markings to delineate distance from point/apex 8 to base face 9 and utilize this value to understand how far the wedge 1 has been inserted into a joint.

The projection coupling 6 is detailed to include threads 11 that can couple with corresponding female threaded elements of mating/corresponding diameter and pitch. The length of the projection 6 is such that the mating female couplings will be secure for at least some adequate distance when affixed to corresponding female threaded elements. In some embodiments, the projection 6 is long enough that it extends proximally beyond the corresponding mating female guide coupling a sufficient length that it can also be secured to the mating female handle coupling (so that converting between the first/positioning and second/cutting configurations is accomplished by installing or removing the handle relative to the wedge when the cutting guide is already mounted to it). The socket 21 typically includes an extension portion that is configured with a size and depth to receive the portion of the threaded wedge coupling bolt 6 that extends proximally beyond the guide coupling nut 20 (see FIG. 15). While this projection 6 is an example embodiment of the coupling part 6 of the wedge 1, it is not required for function. It may also take a variety of forms, for example square or triangular shaped.

It will be understood that the wedge can be provided in other embodiments that provide the described functionality. For example, the wedge coupling part can be other than a threaded projection, such as a threaded female aperture (where mating coupling parts of the handle and the cutting guide are provided by threaded projections), by a male projection or other element that is not threaded but has another type of securement (a lateral through-hole that receives a pin), or by another conventional coupling part. In addition, the wedge can have other dimensions and/or aspect ratios to provide the same wedging and bone-repositioning functionality described herein.

Referring to FIGS. 1 and 6-10, the handle 12 includes an extension member 12a and a head 13. The extension member 12a is elongated and has a coupling part 14 at its distal end. The handle coupling 14 mates with the wedge coupling 6 to removably mount the handle 12 to the wedge 6 in the first system configuration to form the joint-repositioning tool 100a (see FIG. 10). For example, the handle coupling 14 can be a threaded aperture formed into the distal end surface of the handle 12, with the female threads mating with the male threads of the wedge projection 6. Also, the head 13 is positioned at the proximal end of the handle 12 with the extension member 12a extending distally from it. The head 13 is typically enlarged, that is, larger in area in a longitudinal/axial view than the extension member 12a. The enlarged head 13 provides added weight for leverage and/or a strike face for hitting (e.g., with a mallet) to drive the wedge 1 into the joint during surgery. Further, in some embodiments such as that depicted, the head 13 can include a socket 21 (e.g., with internal polygonal sides) for matingly receiving a locking nut 20 (e.g., with external polygonal sides), which has an internally threaded through-hole 22 that mates with the threaded projection 6 of the wedge 1, so that the socket 21 of the handle 12 can be used to tighten and loosen the nut 20 onto and off of the wedge projection 6. The socket 21 typically includes an extension portion that is configured with a size and depth to receive the portion of the threaded wedge coupling bolt 6 that extends proximally beyond the guide coupling nut 20 (see FIG. 15).

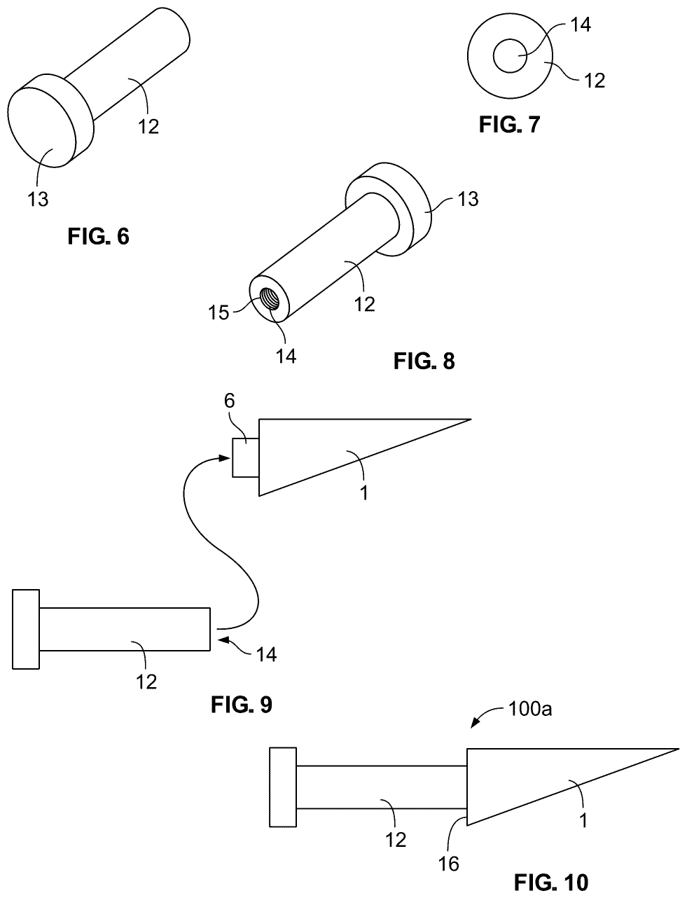

Referring specifically to FIG. 6, in the depicted embodiment, the extension member 12a and the head 13 are each generally cylindrical in shape. The cylindrical extension member 12a can typically have a length selected for manually grasping during use without interfering with the placement of the wedge 1, for example between 60 and 150 millimeters. The cylindrical extension member 12a can typically have a diameter selected for manually grasping during use, for example between 8 and 20 millimeters.

Referring specifically to FIGS. 7-8, in the depicted embodiment, the threaded aperture coupling 14 can be centrally formed in a distal end surface of the handle 17 opposite the enlarged head 13 (which is at the proximal end of the handle 17). The threaded aperture coupling 14 can be recessed extending for a sufficient distance within the extension member 12a to enable mating attachment to the threaded projection/extension coupling 6 of the wedge 1.

It will be understood that the handle can be provided in other embodiments that provide the described functionality. For example, the handle coupling part can be other than a threaded female element, such as a threaded male element (where the coupling part of the cutting guide is of the same type and the mating coupling part of the wedge is a threaded aperture), by a female element that receives the wedge projection but is not threaded and instead has another type of securement (a lateral through-hole that aligns with a through-hole in the wedge projection and receives a pin), or by another conventional coupling part. In addition, the handle can have other dimensions and/or aspect ratios to provide the same grasping and force-applying functionality described herein.

Referring specifically to FIGS. 9-10, the wedge 1 and the handle 12 removably mount together, by the mating connection of the wedge coupling part 6 and the handle coupling part 14, in the first system configuration to form the joint-repositioning surgical tool 100a. In the depicted embodiment, the threaded projection coupling 6 of the wedge 1 is received and secured in the threaded aperture 14 of the handle 12 to assemble these components together at junction 16. It will be understood that these are an example coupling parts that can be included to removably mount the wedge and handle together to form the joint-repositioning tool 100a with the wedge leading tip 8 and the enlarged head 13 opposite each other. The enlarged head 13 can be hit with a mallet or other large tool while the wedge 1 is inserted into a joint space. This advances the wedge 1 forward. While this embodiment anticipates use of large impaction forces, it should be understood that this is optional and not a requirement for function.

Referring to FIGS. 1 and 11-15, the cutting guide 17 includes a block 17a and a guide coupling part 20, with the guide block 17a having a plurality of guide slots 18 formed in it. The guide block 17a can be a plate (or other body), with a rectangular (or other regular or irregular) shape. The guide slots 18 are linear (e.g., extending vertically in typical surgical-use orientations, as depicted), sized to receive conventional surgical orthopedic saw blades all the way through them, and spaced apart a distance selected to provide bone cuts at two adjacent ends of two bones in the same joint. The guide coupling 20 mates with the wedge coupling 6 to removably mount the cutting guide 17 to the wedge 6 in the second system configuration to form the surgical saw-guide tool 100b (see, e.g., FIG. 15). For example, the guide block 17a can have an aperture 19 extending all the way through it that receives the threaded wedge projection 6 all the way through it, and the guide coupling 20 can be a nut with a threaded opening 22, with its female threads mating with the male threads of the wedge projection 6.

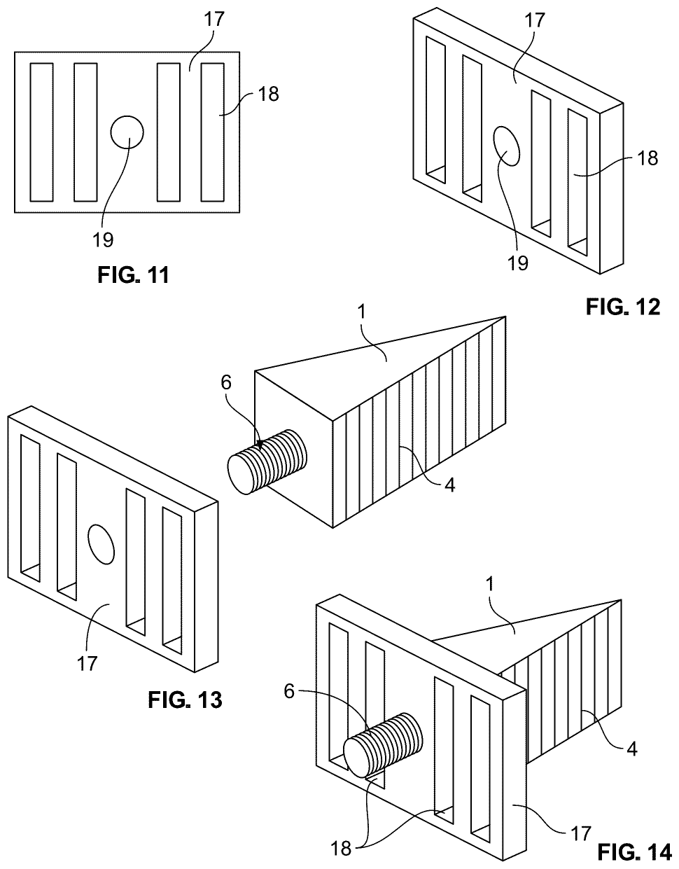

Referring specifically to FIGS. 11-12, in the depicted embodiment, the threaded aperture coupling 19 is positioned centrally in the guide block 17a and between at least two of the guide slots 18. The threaded aperture coupling 19 is slightly larger than the diameter of threaded wedge projection 6. There can be four or another number of the guide slots 18, each extending all the way through the guide block 17a from one side to the other so that the bone saw blades can pass through. To accommodate receiving the bone saw blade through them, the guide slots 18 can be for example 1 to 2 millimeters in width. The guide slots 18 are typically parallel to each other and extend perpendicularly through the guide block 17a.

It will be understood that the cutting guide and/or the component couplings can be provided in other embodiments that provide the described functionality. For example, the guide coupling part can be other than a female-threaded nut, such as a threaded male element that extends from the guide block in place of the aperture (where the coupling part of the handle guide is of the same type and the mating coupling part of the wedge is a threaded aperture), by a structure with an opening that receives the wedge projection but is not threaded and instead has another type of securement (a lateral through-hole that aligns with a through-hole in the wedge projection and receives a pin), or by another conventional coupling part (e.g., clamps, handles, bushings, clips, or other conventional fasteners). Thus, in a vice versa arrangement, the wedge coupling is an aperture and the mating handle and cutting guide couplings are each a mating projection. Accordingly, the wedge coupling part, the handle coupling part, and the cutting guide coupling part typically include at least one projection and at least one aperture that mate with each other, for example with the wedge coupling part including one of a projection and an aperture and the handle coupling part and the cutting guide coupling part each including the other of a projection and an aperture.

In addition, the cutting guide can have other shapes and/or sizes. Further, at least two of the guide slots can be angled relative to each other (non-parallel) and/or at least one of the guide slots can be extend non-perpendicularly through the guide block, so the guide slot that best enables the desired correction can be selectively used by the orthopedic surgeon.

FIGS. 13-15 show the wedge 6 and the cutting guide 17 being assembled together into the second system configuration to form the saw-guide tool 100b. FIG. 13 shows the wedge projection 6 aligned with the guide aperture 19, FIG. 14 shows the wedge projection 6 extending through the guide aperture 19, and FIG. 15 shows the fully assembled saw-guide tool 100b including the wedge 1 and the cutting guide 17. Also shown is the wedge projection 6 extending through the guide aperture 19 and protruding sufficiently far therethrough so that the guide coupling 20 can be fastened onto the projection to secure the wedge 6 and the cutting guide 17 together in the second configuration. Further shown is a typical spatial arrangement of the guide slots 18 in the cutting guide 17, with the most-central of the slots 18 arranged so that a bone saw blade extending through them would not collide/interfere with the wedge 1 during surgical use of the saw-guide tool 100b. That is, the slots 18 are located through the cutting guide 17 with the central-most slots configured to be dimensionally spaced just outside of the dimension of the base face 9 of the wedge 1. For example, if the lateral dimension of the base face 9 is 10 millimeters, the lateral distance from the center of the cutting guide 17 to the center of the most-central slot would be not less than 6 millimeters, as the slot center to center would be 12 millimeters, which is greater than the 10 millimeter face 9.

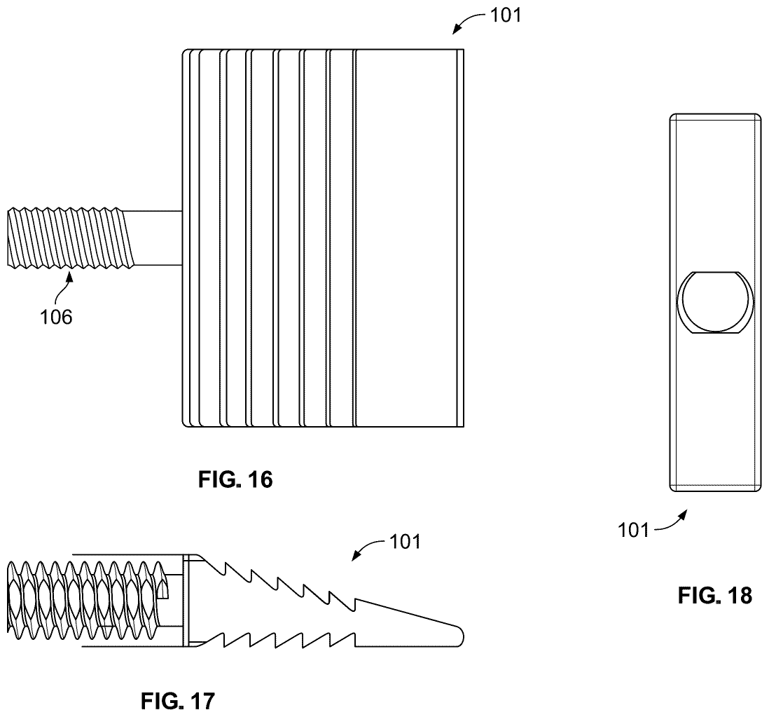

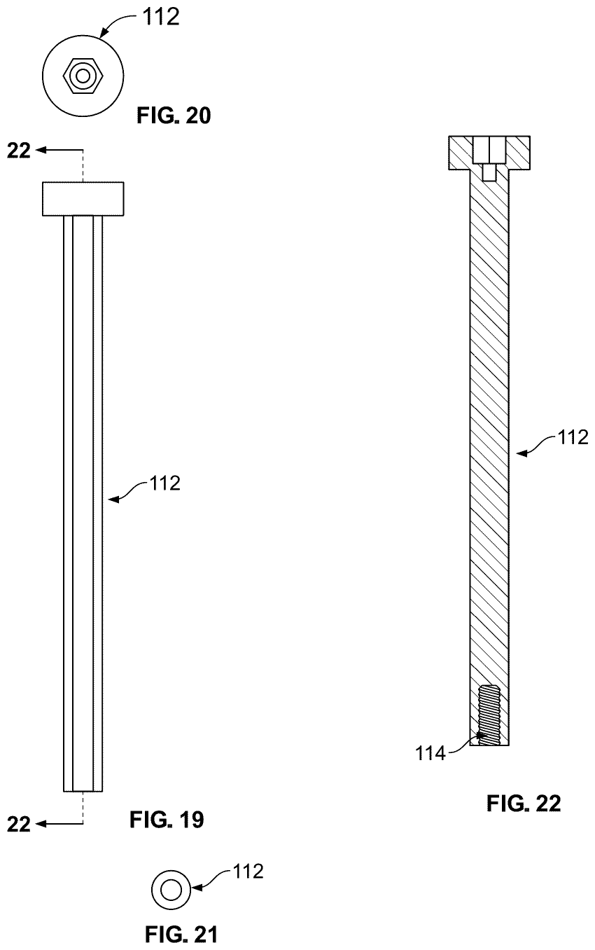

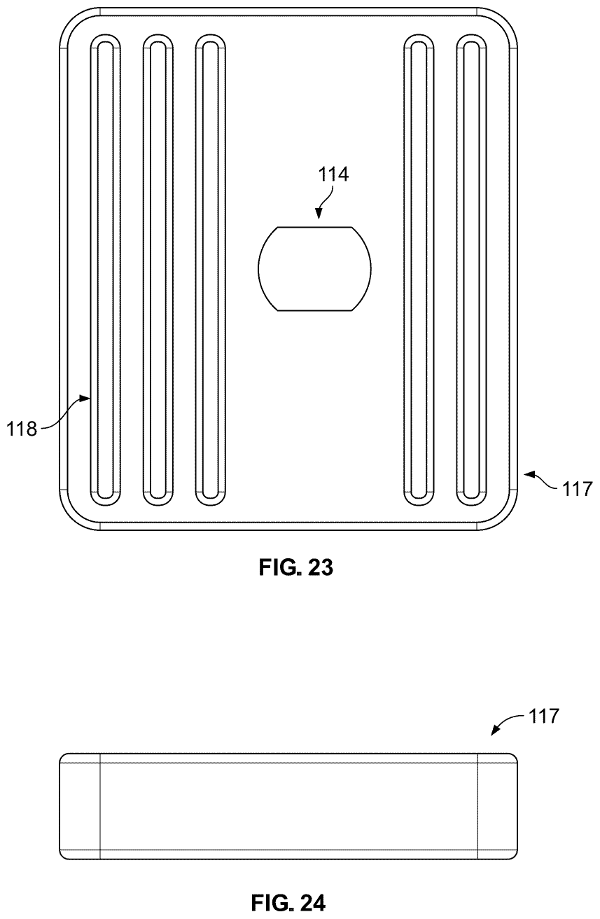

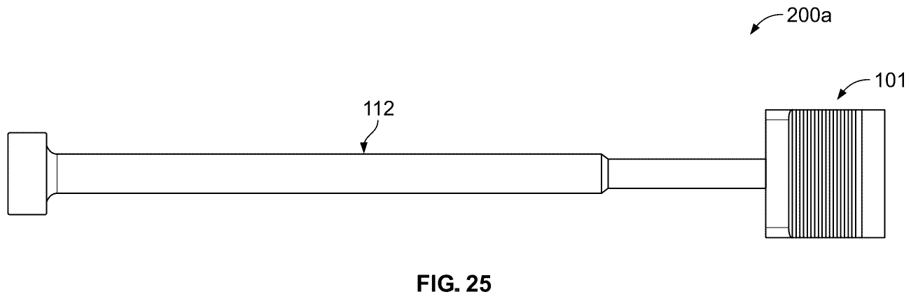

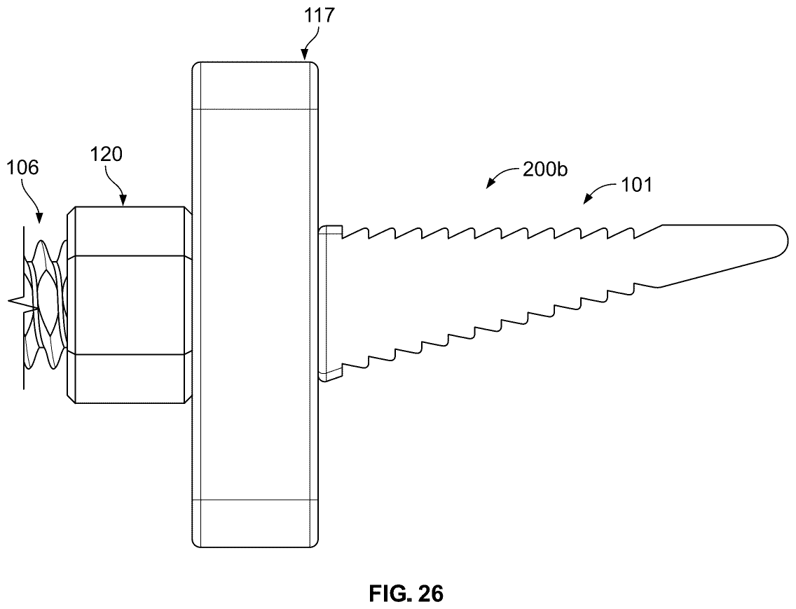

Referring now to FIGS. 16-28, there is shown an orthopedic surgical device system for reducing angular deformities in joints according to a second example embodiment. This embodiment is substantially similar to the embodiment described above, thus it includes a handle 112 and a cutting guide 117 that are each removably mountable to a wedge 101. Similarly, these components of the system are reconfigurable between a first/positioning configuration with the handle 112 mounted to the wedge 101 to form a joint-repositioning tool 200a (see FIG. 25), and a second/cutting configuration with the cutting guide 117 mounted to the wedge 101 to form a saw-guide tool 200b (see FIG. 26). The wedge 101, the handle 112, and the cutting guide 117 each include a coupling part that enables this reconfiguring, for example the wedge coupling can be a threaded projection 106, the cutting guide coupling can be a threaded nut 120, and the handle coupling can be a threaded aperture.

In this embodiment, the wedge 101, handle 112, and cutting guide 117 include design updates, including dimensional and aspect ratio changes, but they provide the same essential functionality as the embodiment described above, except as noted herein.

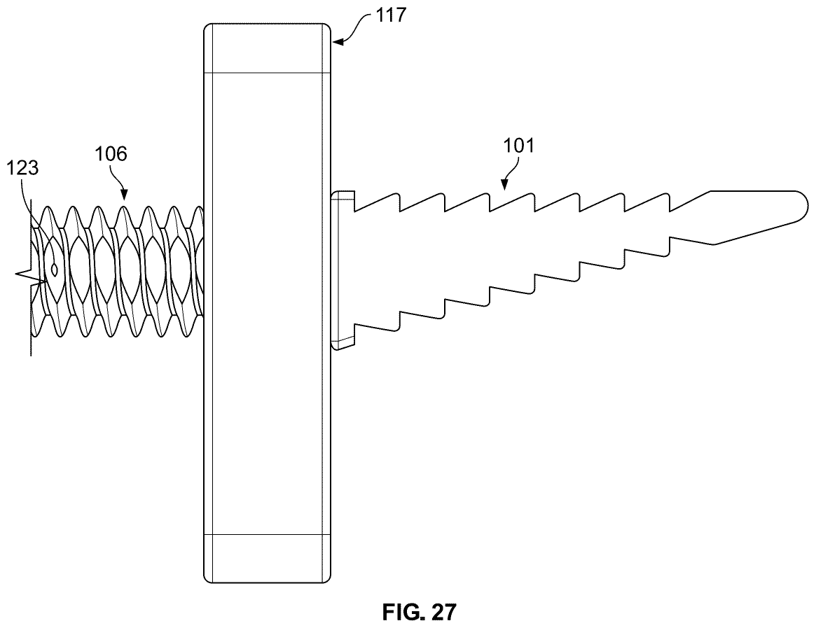

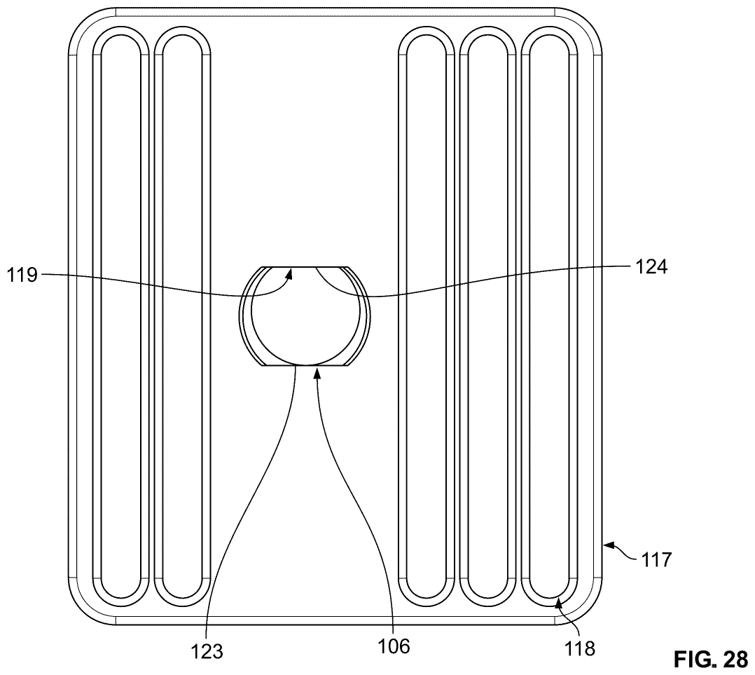

In particular, FIGS. 27-28 in particular show that the wedge 101 mounts perpendicularly to the cutting guide 117, with the cutting guide having a flat distal surface abutting the flat base/proximal face of the wedge. Also, the guide slots 118 extend perpendicularly through the cutting guide 117 and thus guide the orthopedic saw blades in a plane parallel to the axis of the elongate handle 112. This is all consistent with the previously described embodiment.

However, in this embodiment, the cutting guide 117 and the wedge 101 removably mount together in an angularly fixed orientation that does not permit rotational adjustment of the cutting guide 117 about the longitudinal axis of the handle 112. For example, in the depicted embodiment, the wedge coupling 106 is a threaded projection that is cylindrical except with at least one flattened side (e.g., two flattened sides are depicted) 123, that is, the helical threading that extends radially outward from the core of the projection is truncated or excluded at that arc segment of the projection. Also, the cutting guide opening 119 has at least one flattened side (e.g., two flattened sides are depicted) 124, with the guide opening 119 configured to receive the wedge projection 106 therethrough. The guide opening 119 thus has the same shape as the wedge projection 106, with slightly larger dimensioning to allow the wedge projection 106 to be inserted into the guide opening 119 without undue frictional resistance but without excess clearance to provide a minimum clearance fit. The conforming flattened sides 123 and 124 of the wedge projection 106 and the guide opening 119 define a fixed angular position of the cutting guide 117 relative to the wedge 101 when the two components are mounted together to form the joint-repositioning tool 200a.

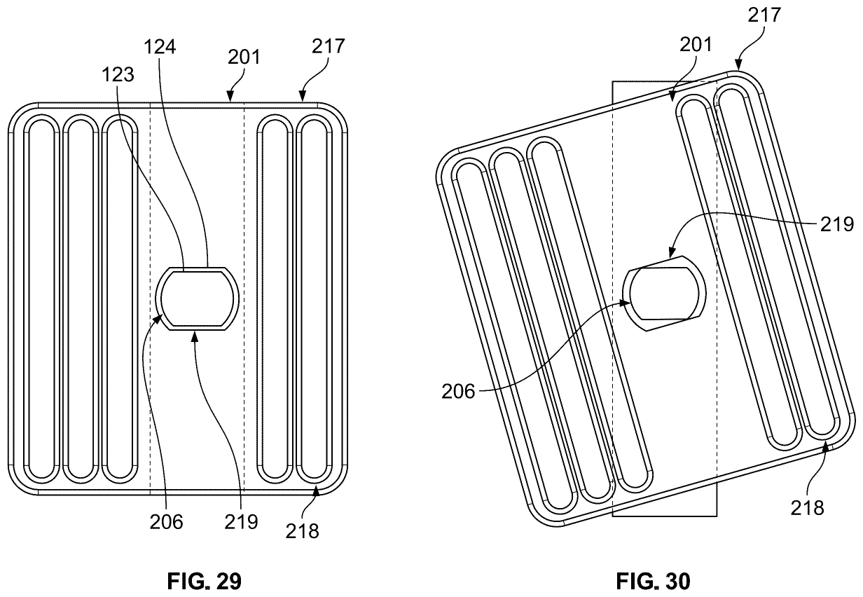

Referring now to FIGS. 29-30, there is shown a wedge 201 and cutting guide 217 of an orthopedic surgical device system for reducing angular deformities in joints according to a third example embodiment. This embodiment is substantially similar to the embodiments described above, thus it includes a handle (not shown) and the cutting guide 217 that are each removably mountable to the wedge 201 to be reconfigurable between a first/positioning configuration with the handle mounted to the wedge 201 to form a joint-repositioning tool and a second/cutting configuration with the cutting guide 217 mounted to the wedge 201 to form a saw-guide tool. The wedge 201, the handle, and the cutting guide 217 each include a coupling part that enables this reconfiguring, for example the wedge coupling can be a threaded projection 206 and the cutting guide 217 can include an opening 219 that receives the wedge projection 206.

In this embodiment, the wedge 201, handle, and cutting guide 217 have substantially the same design and function as described above, except as noted herein.

In particular, as in the second embodiment, the wedge coupling 206 is a threaded projection that is cylindrical except with at least one flattened side (e.g., two flattened sides are depicted) 223, and the cutting guide opening 219 has at least one flattened side (e.g., two flattened sides are depicted) 224, with the guide opening 219 configured to receive the projection 206 therethrough (the guide opening 219 has the same shape as the wedge projection 206).

In this embodiment, however, the guide opening 219 is oversized relative to the wedge projection 206 to provide a loose fit between the parts such that the cutting guide 217 can be moved slightly relative to the wedge 201 in order to fine-tune the position and orientation of the guide slots 218 after the wedge 201 has been inserted into the joint during surgery (to avoid having to remove and reinsert the wedge 201). Once the cutting guide 217 is in the precise desired position, it can be fixed in place there by the threaded nut or other guide coupling (not shown). In this way, the cutting guide 217 can be moved slightly relative to the wedge 201 in a transverse direction (within the plane of the cutting guide 217; see FIG. 30) to enable positioning and orienting the guide slots 218 closer to or farther from the bone end.

Furthermore, the cutting guide 217 can be moved slightly relative to the wedge 201 in an angular/rotational direction about the wedge projection 206 (see FIG. 30) to enable positioning and orienting the guide slots 218 at an angle (relative to the footprint defined by the base face of the wedge 201). This angled bone cutting can also be useful in making corrections of biplanar deformities, with the two adjacent bones not necessarily being aligned with each other before cutting.

In the depicted embodiment, the guide opening 219 is not oversized sufficiently to allow the cutting guide 217 free rotation of 360 degrees about the wedge projection 206. Rather, the guide opening 219 is oversized relative to the wedge projection 206 to permit rotation of an acute angle (e.g., up to 10 degrees) before interference between the flattened sides 123 and 124 of the wedge projection 206 and the guide opening 219 prevents further rotation.

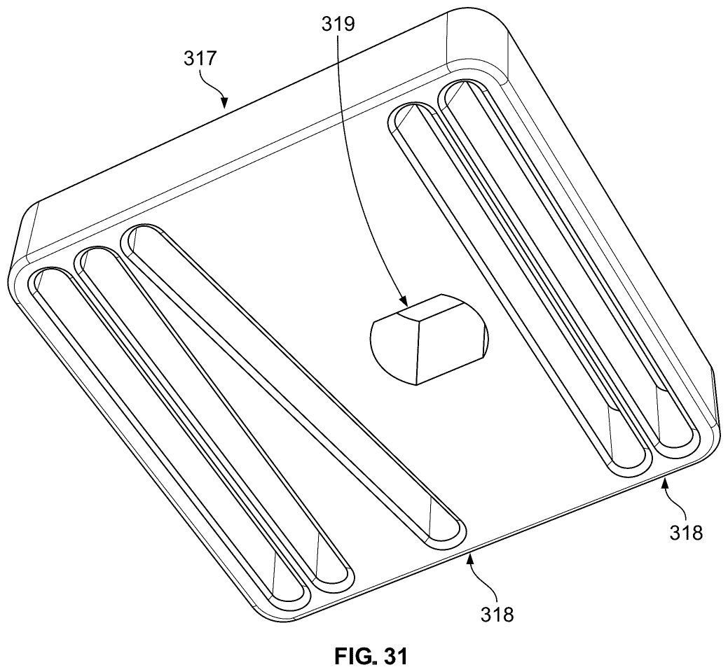

Referring now to FIGS. 31-33, there is shown a cutting guide 317 and a wedge 301 of an orthopedic surgical device system for reducing angular deformities in joints according to a fourth example embodiment. This embodiment is substantially similar to the embodiments described above, thus it includes a handle (not shown) and the cutting guide 317 that are each removably mountable to the wedge 301 to be reconfigurable between a first/positioning configuration with the handle mounted to the wedge 301 to form a joint-repositioning tool (as depicted) and a second/cutting configuration with the cutting guide 317 mounted to the wedge 301 to form a saw-guide tool. In this embodiment, the wedge 301, handle, and cutting guide 317 have substantially the same design and function as described above, except as noted herein.

In this embodiment, the cutting guide 317 has a plurality of the guide slots 318, with at least one of the guide slots 318 being angled relative to at least one other of the guide slots. That is, the depicted four parallel guides slots 318 are vertical in typical orthopedic surgical uses, and the angled guide slot 318 is angled from vertical in typical orthopedic surgical uses.

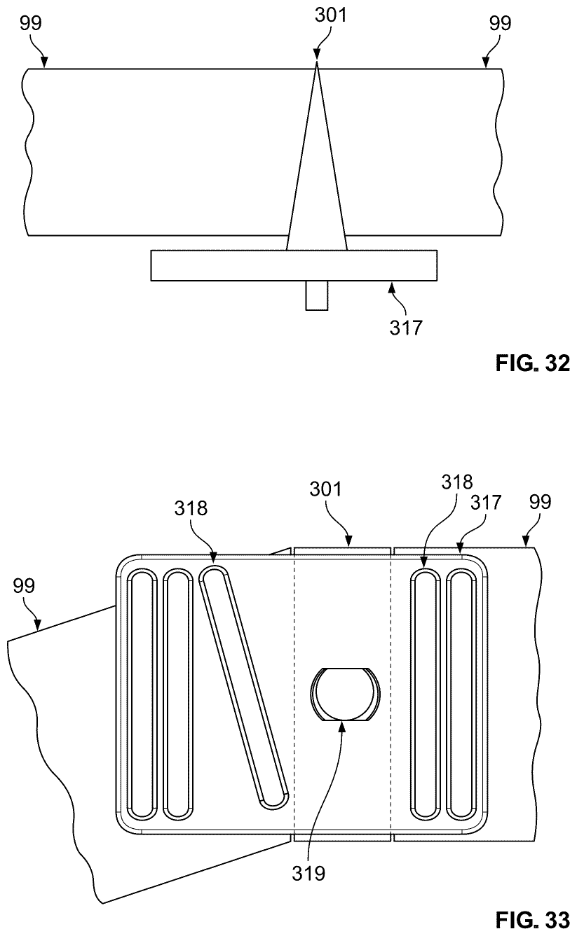

This angled guide slot 318 can be used for orthopedic surgeries involving deformity correction of the ends of two bones 99 in a joint, with the two adjacent bones not necessarily being aligned with each other before cutting. As shown in FIG. 33, a saw blade can be inserted through either of the vertical guide slots 318 to the right of the guide opening 319 to remove the end of the corresponding bone 99 (at the right side of the figure). And the same or another saw blade can be inserted through the angled guide slot 318 to the left of the guide opening 319 to remove the end of the other bone 99 (at the left side of the figure). This results in the two bones 99 now having newly formed end edges that are substantially parallel to each other in this side view to correct the deformity in a first plane. To correct the deformity in a second perpendicular plane, reference is made to FIG. 32, which shows the triangular shape of the wedge 301 inserted in the joint to reposition the left-side bone portion 99 into substantial alignment with the right-side bone portion so that the same two cuts make the newly formed bone edges substantially parallel to each other in this top view to correct the deformity in a second plane that is perpendicular to the first plane.

It should be noted that the opening 319 of the cutting guide 317 need not be centered (for example, as depicted in this embodiment). Also, the opening 319 of the cutting guide 317 can be oversized, as in the previous embodiment, to provide an acute angle of rotation for fine-tuning adjustment of the slot position/orientation before making the cuts.

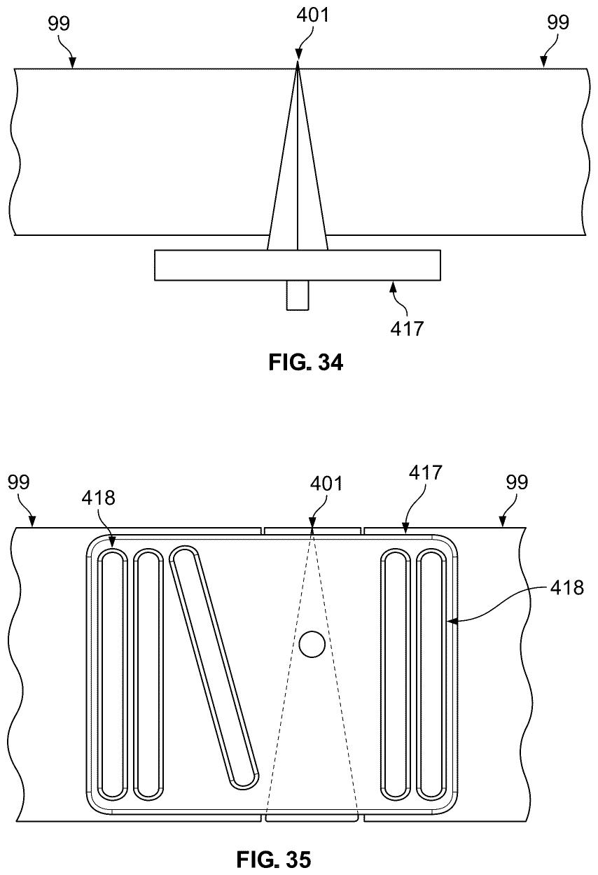

Referring now to FIGS. 34-35, there is shown a cutting guide 417 and a wedge 401 of an orthopedic surgical device system for reducing angular deformities in joints according to a fifth example embodiment. This embodiment is substantially similar to the embodiments described above, thus it includes a handle (not shown) and the cutting guide 417 that are each removably mountable to the wedge 401 to be reconfigurable between a first/positioning configuration with the handle mounted to the wedge 401 to form a joint-repositioning tool (as depicted) and a second/cutting configuration with the cutting guide 417 mounted to the wedge 401 to form a saw-guide tool. In this embodiment, the wedge 401, handle, and cutting guide 417 have substantially the same design and function as described above, except as noted herein.

In this embodiment, the wedge 401 has a truncated shape, that is, it's triangular in two intersecting planes. Thus, the wedge 401 has a triangular shape in a first plane when viewed from the top (FIG. 34) and it also has a triangular shape in a second plane when viewed from the side (FIG. 35), as opposed to the previous embodiments in which it has a rectangular shape in a side view (e.g., FIG. 33).

This truncated wedge 401 can be used for orthopedic surgeries involving bi-planar deformity correction of the ends of two bones 99 in a joint, in which the end of a bone needs to be cut in two different planes. As shown in the top view of FIG. 34, the truncated wedge 401 is triangular-shaped in a first plane so that when it's inserted in the joint it repositions the left-side bone portion 99 into substantial alignment with the right-side bone portion in a horizontal plane. And as shown in the side view of FIG. 35, the truncated wedge 401 is also triangular-shaped in a second perpendicular plane so that when it's inserted in the joint it repositions the left-side bone portion 99 into substantial alignment with the right-side bone portion in a vertical plane. With the bone 99 now aligned in both planes, a saw blade can be inserted through one of the vertical guide slots 418 to the right of the guide opening 419 to remove the end of the corresponding bone 99 (at the right side of FIG. 35) and inserted through one of the vertical guide slots 418 to the left of the guide opening 419 to remove the end of the corresponding bone 99 (at the left side of FIG. 35). This results in the two bones 99 now having newly formed end edges that are substantially parallel to each other in both the horizontal plane (as shown in the top view) and the vertical plane (as shown in the side view).

In some embodiments, the cutting guide is mounted to the wedge, without the guide coupling being mounted to the wedge coupling. The handle is then mounted to the wedge, with the cutting guide still mounted to the wedge, with the handle coupling mounted to the wedge coupling. In this third configuration of the system, the cutting guide is secured in place between the wedge and the handle, with this configuration forming a positioning and cutting tool that can be used for both functions without converting between the first/positioning and second/cutting configurations. Also, in use of the system in this third configuration with all three components assembled together, the wedge can be inserted securely into the joint, then the handle removed for the needed bone cutting in the second configuration (leaving the cutting guide mounted to the wedge), and then the handle reattached for removal of the wedge (and gripped section bone) in the third configuration. In some such embodiments, the cutting guide and the wedge are fixedly attached together and not removable from each other by the user during the normal intended use of the system as described herein.

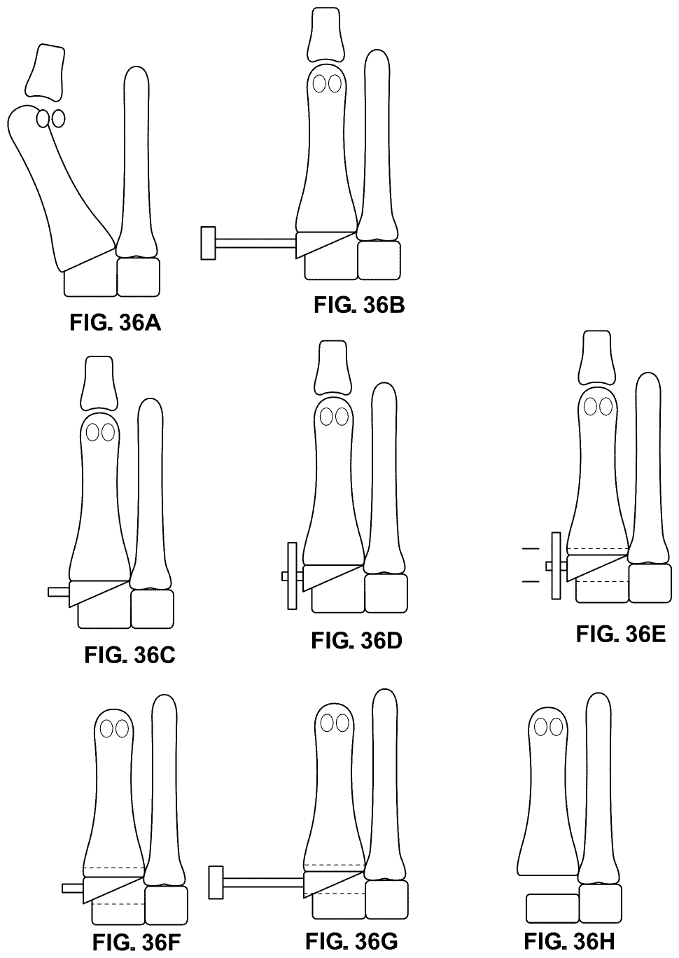

Referring now to FIGS. 36A-36H, there is shown a method of correcting orthopedic deformities according to an example embodiment of the invention. The method can be implanted using the orthopedic surgical systems described herein and/or orthopedic surgical systems of other designs that provide substantially the same functionality. For illustration purposes, however, the method will be described with primary reference to the first example embodiment of FIGS. 1-15.

In the course of surgical fixation, anatomic dissection is carried out and the bone is exposed in a standard surgical manner. When it is determined that angular deformity correction will be performed by corrective osteotomy and joint arthrodesis (FIG. 16A), orthopedic surgical devices are used to aid in anatomic reduction and precision bone cuts. The orthopedic surgical system is brought into the surgical field and assembled into a joint-repositioning tool with a handle 12 screwed or otherwise coupled to a wedge 1. Depending on right side or left side foot, the tool is used either right side up or upside down, for example with the base face of the wedge facing proximally, and with the leading tip facing distally. After the joint ligaments are sectioned making the joint more mobile, the wedge 1 is placed into the now mobile joint. The surgeon uses a mallet or other force-applying device to force the wedge 1 into the joint, for example by striking the head of the handle 12 of the tool with the mallet. As the wedge 1 is advanced into the joint, reduction of the preoperative deformity angle will be appreciated. Further advancement of the wedge 1 creates further reduction, up to the degree of the wedge leading tip when it approaches the furthest aspect of the joint (FIG. 36B).

The handle 12 can then be detached from the wedge 1 (FIG. 36C). Serrations on the side surfaces of the wedge 1 dig into the cartilage on adjacent sides of the joint, aiding in stability of the wedge 1 after handle 12 removal. The surgeon has the advantage of reducing the deformity slowly to any degree he or she determines to be reduced enough or completely. Lesser insertion of the wedge 1 is permitted if the surgeon deems the deformity to be reduced, that is, the wedge can be inserted only partially into the joint instead of all the way through it, if that best achieves deformity reduction.

In typical embodiments, the tool can be used to not only reduce the angular deformity to anatomic standards and maintain this correction, but also to permit precision bone cuts that will maintain this correction once the cut bone is removed. As such, with the handle 12 removed, a corresponding cutting guide (i.e., a saw fence block) 17 with slots (e.g., parallel) for a saw blade is brought to the operating field. On the exposed proximal side of the wedge 1 is a coupling (e.g., a threaded projection) that the handle 12 was removably coupled to (by a threaded aperture or other coupling of the handle). The cutting guide 17 also removably couples to the wedge 1, for example the cutting guide 17 can have an opening (e.g., a hole or slot) that receives the same wedge threaded projection, and a threaded nut or other guide coupling can then be mounted to the exposed end of the wedge threaded projection to secure the cutting guide 17 to the wedge 1. The cutting guide 17 is affixed then to the wedge 1 by way of this projection and nut/fitting, stabilizing the cutting guide 17 in place to form a saw-guide tool (FIG. 36D). Optionally, the cutting guide can have a prescribed angle selected to affix it to the wedge.

In example embodiment, the orthopedic surgical system and method is both simple and secure. The wedge 1 is both friction and mechanically secured within the joint by way of inherent pressure between two bones and serrations on the wedge 1 that dig into the softer cartilage within. With the cutting guide 17 secured on the inserted wedge 1, the surgeon now has both hands free to precisely cut the adjacent bones. Thus, once the cutting guide 17 is secured, the surgeon then can use a bone saw of their choosing with a saw blade of sufficient length to insert through selected guide slots of the cutting guide 17 and engage the bones on the other side. The surgeon will choose a guide slot corresponding to their desired level of bone cut, often determined by length relationships of the adjacent bones, sufficient enough to fully remove the cartilage but without excessive bone removal. As both bone cuts are made on either side of the wedge 1, it will become immediately obvious that these are parallel cuts with a reduced deformity, making at least one of the bone cuts effectively angular within the bone (FIG. 36E).

After establishing and completing these bone cuts, the tool is then removed sequentially by first removing the cutting guide 17 from the wedge 1 (FIG. 36F), then reattaching the handle 12 to the wedge 1 (FIG. 36G), and manipulating the wedge 1 backwards until it is fully extruded/retracted from the joint. After establishing and completing these bone cuts, the tool is then removed sequentially by first removing the cutting guide 17 from the wedge 1 (FIG. 36F), then reattaching the handle 12 to the wedge 1 (FIG. 36G), and manipulating the wedge 1 backwards until it is fully extruded/retracted, with the serrations dug into the joint cartilage and/or bone also retracting the adjacent cut bone pieces. Remaining behind are two parallel cut bones that when approximated will be flush only at the exact angle of reduction (FIG. 36H).

The above is a more general description of example methods according to various embodiments of the invention, and the below is a more detailed description of example methods according to various embodiments of the invention.

This example method is designed to facilitate rapid deformity reduction using a wedge and cutting guide. Relatively small deformities can be corrected using relatively small-angled wedges and relatively large deformities corrected using relatively large-angled wedges. Also, in addition to aligning the bones and making bone cuts that are strictly vertical (as described above), the method can include reducing (but not eliminating) the bone misalignment and making angled bone cuts using rotatable cutting guides 218 and/or angle-slotted cutting guides 318. Furthermore, in addition to correcting mono-planar deformities (as described above), the method can include correcting biplanar deformities using angle-slotted cutting guides 317, or using parallel-slotted cutting guides 417 in combination with truncated wedges 401.

1. Joint exposure is carried out in standard methods by the surgeon including soft tissue retraction, ligament severance, and removal of bone material such as bone spurs or scar tissue that would impede direct joint access.

2. The orthopedic surgical system is brought into the surgical field. The wedge is secured to the handle. The wedge leading angle may be large or small, and the correct wedge is chosen based on the predetermined range of deformity angle to be reduced.

3. The leading tip/edge of the wedge is approximated with the exposed joint with the 90-degree angle edge of the wedge facing distally or towards the farther bone to be reduced in a first plane. It should be noted that if the surgeon chooses a truncated wedge for biplanar correction, then the block is also inserted and arranged for a second plane of reduction. For example, if plantarflexion or more declination of the distal bone segment is desired, the wedge would be inserted with the wider portion facing upward and the narrower portion facing downward.

4. A mallet or other object of sufficient mass is used to advance the wedge into the joint. The surgeon will determine the required depth of insertion when the pre-surgical deformity has been reduced by the desired amount. The wedge's proximally oriented serrations dig into the joint cartilage and/or bone to secure the wedge in place to the bones.

5. Once the wedge has been inserted to the desired depth, the handle is then disconnected from the wedge while the wedge remains inside the joint.

6. A cutting guide is then brought into the surgical field and attached to the inserted wedge. This will serve as a saw fence for upcoming bone cuts. The cutting guide may consist of parallel slots on either side of the attachment for parallel cuts on both separated bones. Alternatively, a cutting block with angled slots on one or both sides of the attachment may be used if the surgeon chooses to correct in a second plane (FIGS. 31-33).

7. Slight enlargement of a connection opening in the cutting guide can be included to provide for some limited rotation of the cutting angle when the articulation is not at 90 degrees to the wedge face. For example, if the surgeon determines the true joint angle and the wedge position are not parallel, he or she may rotate the cutting guide to match the true joint angle (FIGS. 29-30).

8. The cutting guide is secured to the wedge by using a nut (or other guide fitting/coupling) that is fastened to the threaded projection (or other wedge coupling) after the cutting guide is positioned on the wedge. This nut can be tightened to the wedge using the head of the handle, where a polygonal (e.g., hexagonal) socket can be located to accommodate the nut and projection, or using another device. The handle is rotated while the nut advances over the threaded projection until the cutting guide is secured at the desired rotational position.

9. The handle head is then removed from the nut while the cutting guide and wedge remain secured in the joint.

10. A bone saw with a blade appropriate for the cutting guide slots is brought into the field.

11. Bone cuts are made on adjacent sides of the attachment (of the cutting guide to the wedge) to incise one or both ends of the bone segments. Cuts are typically made to a sufficient depth to fully appreciate the plane and angle of the cut if the blade is not long enough and without fully separating the bone ends, however this is not required. These cuts may be completed after the tool is removed.

12. Once the surgeon has completed the bone cuts, the tool is removed in reverse order: the handle head (or another device) is used to unfasten the locking nut, the cutting guide is slid off the projection, the handle is re-attached to the projection, and the wedge is manually extracted from the now prepared joint.

13. Because of the wedge's proximally oriented serrations, the bone ends are extruded/retracted with the wedge upon removal of the wedge.

14. The bone ends are then brought into approximation to assess whether a flush fit has been achieved and evaluate the angle of deformity reduction in all planes. Any adjustments can be made using a “feathering” technique with a saw blade at this time.

The joint is then fixed in place using any technique desired by the surgeon and from any point along the bone segments since there has been no adjacent bone violation in preparation for joint fixation.

Accordingly, various embodiments of the system and method provide advantages over prior systems and methods, specifically:
1. A device that can reduce an angular deformity at the joint level of any magnitude regardless of preoperative measurement;
2. A device that consists of a minimal number of components that are simple to assemble as to require minimal to no training or outside assistance;
3. A device that is robust enough to maintain this angular reduction in preparation for bone cutting;
4. A device that requires no ancillary attachment points or adjacent bone anchorage;
5. A device that incorporates a cutting jig/guide/fence such that precision bone cuts can be performed while the angular deformity is in its reduced state, alleviating the concern of over or under correction after the apparatus is removed;
6. A device that is fixation agnostic, permitting any form of acceptable fixation to be used after disassembly;
7. A device that is an inexpensive but effective method for performing angular reduction and bone cutting procedures;
8. A device that does not rely on complex preoperative radiographic planning.

While the invention has been described with reference to example embodiments, it will be understood by those skilled in the art that a variety of modifications, additions and deletions are within the scope of the invention, as defined by the following claims.

What is claimed is:

1. An orthopedic surgical system for reducing an angular deformity in a joint, the system comprising:
a wedge including a wedge block and a wedge coupling part, wherein the wedge block has a triangular shape and includes two opposing side faces that are angled with respect to each other and that each include serrations that dig into joint cartilage to secure the wedge in place to and between two adjacent bones of the joint;
a handle including an elongated extension member and a handle coupling part, wherein the handle coupling part mates with the wedge coupling part to removably mount the handle to the wedge in a first configuration of the system to form a joint-repositioning orthopedic surgery tool; and
a cutting guide including a guide block and a guide coupling part, wherein the guide block includes a plurality of guide slots formed in it that each receive an orthopedic bone saw blade all the way through them, wherein the guide coupling part mates with the wedge coupling part to removably mount the cutting guide to the wedge in a second configuration of the system to form a saw-guide orthopedic surgery tool,
wherein in use, the wedge of the joint-repositioning tool is inserted between the two adjacent bones of the joint to reposition at least one of the bones, and then the cutting guide of the saw-guide tool is used to guide the orthopedic bone saw blade to remove at least one bone end portion, to form bone end edges that are substantially parallel so the bones are now substantially aligned.

2. The orthopedic surgical system of claim 1, wherein the wedge block includes a distal leading tip formed by the two angled side faces, and a proximal base face opposite the leading tip, wherein the wedge coupling is on the base face.

3. The orthopedic surgical system of claim 1, wherein the wedge block further includes two opposing top and bottom faces that are angled with respect to each other and that extend between the two angled side faces to form a truncated wedge shape, wherein in use in the first configuration forming the joint-repositioning orthopedic surgery tool, the truncated wedge repositions at least one of the bones in two planes.

4. The orthopedic surgical system of claim 1, wherein the handle further comprises a proximal head from which the extension member extends, wherein the head is larger in cross-sectional area than the extension member to define a proximal strike surface that can be forcefully impacted to drive the wedge into the joint when the system is in the first configuration forming the joint-repositioning orthopedic surgery tool.

5. The orthopedic surgical system of claim 1, wherein the guide coupling part is positioned between at least two of the guide slots when the cutting guide is mounted to the wedge in the second configuration forming the saw-guide orthopedic surgery tool.

6. The orthopedic surgical system of claim 1, wherein at least two of the plurality of guide slots of the cutting guide are angled with respect to each other, wherein in use, the two adjacent bones need not be aligned with each other when using the saw-guide orthopedic surgery tool to remove at least one bone end portion.

7. The orthopedic surgical system of claim 1, wherein the wedge coupling part, the handle coupling part, and the cutting guide coupling part include at least one projection and at least one aperture that mate with each other.

8. The orthopedic surgical system of claim 7, wherein the guide block includes an opening that receives the projection coupling part.

9. The orthopedic surgical system of claim 8, wherein the guide block opening includes a flattened side and the wedge coupling part includes a projection with a flattened side, wherein in the second configuration the guide block is mounted to the wedge block in a fixed angular position.

10. The orthopedic surgical system of claim 8, wherein the guide block opening includes a flattened side, the wedge coupling includes a projection with a flattened side, and the guide block opening is oversized relative to the wedge projection so that in the second configuration the guide block is rotatable relative to the wedge block by an acute angle and limited from further rotation by interference between the flattened sides of the wedge projection and the guide block opening.

11. A method of reducing an angular deformity in a joint using the orthopedic surgical system of claim 1, the method comprising:
configuring the system into the first configuration with the handle mounted to the wedge to form the joint-repositioning tool;
inserting the wedge of the joint-repositioning tool between the two adjacent bones of the joint to reposition at least one of the bones and to secure the wedge in position with the serrations dug into the joint cartilage;
reconfiguring the system into the second configuration, while the wedge remains secured in the joint, with the cutting guide mounted to the wedge to form the saw-guide tool; and
inserting the orthopedic bone saw blade through at least one of the guide slots and to remove at least one bone end portion without the need for any additional securement to the two adjacent bones.

12. An orthopedic surgical system for reducing an angular deformity in a joint, the system comprising:
a wedge including a wedge block and a wedge coupling part, wherein the wedge block has a triangular shape and includes two opposing side faces that are angled with respect to each other and that each include serrations that dig into joint cartilage to secure the wedge in place to and between two adjacent bones of the joint, wherein the wedge block includes a distal leading tip formed by the two angled side faces and a proximal base face opposite the leading tip, wherein the wedge coupling is on the base face;
a handle including an elongated extension member and a handle coupling part, wherein the handle coupling part mates with the wedge coupling part to removably mount the handle to the wedge in a first configuration of the system to form a joint-repositioning orthopedic surgery tool, wherein the handle further comprises a proximal head from which the extension member extends, wherein the head is larger in cross-sectional area than the extension member to define a proximal strike surface that can be forcefully impacted to drive the wedge into the joint when the system is in the first configuration forming the joint-repositioning orthopedic surgery tool; and
a cutting guide including a guide block and a guide coupling part, wherein the guide block includes a plurality of guide slots formed in it that each receive an orthopedic bone saw blade all the way through them, wherein the guide coupling part mates with the wedge coupling part to removably mount the cutting guide to the wedge in a second configuration of the system to form a saw-guide orthopedic surgery tool,
wherein the wedge coupling part, the handle coupling part, and the cutting guide coupling part include at least one projection and at least one aperture that mate with each other, and
wherein in use, the wedge of the joint-repositioning tool is inserted between the two adjacent bones of the joint to reposition at least one of the bones, and then the cutting guide of the saw-guide tool is used to guide the orthopedic bone saw blade to remove at least one bone end portion, to form bone end edges that are substantially parallel so the bones are now substantially aligned.

13. The orthopedic surgical system of claim 12, wherein the wedge block further includes two opposing top and bottom faces that are angled with respect to each other and that extend between the two angled side faces to form a truncated wedge shape, wherein in use in the first configuration forming the joint-repositioning orthopedic surgery tool, the truncated wedge repositions at least one of the bones in two planes.

14. The orthopedic surgical system of claim 12, wherein at least two of the plurality of guide slots of the cutting guide are angled with respect to each other, wherein in use, the two adjacent bones need not be aligned with each other when using the saw-guide orthopedic surgery tool to remove at least one bone end portion.

15. The orthopedic surgical system of claim 12, wherein the wedge coupling part includes a projection, the guide coupling part includes an aperture that receives the wedge projection in the second configuration, and the guide block includes an opening that receives the wedge projection through it with the guide block between the wedge block and the guide coupling part in the second configuration, wherein the guide block opening includes a flattened side, the wedge projection includes a flattened side, and the guide block opening is oversized relative to the wedge projection so that in the second configuration the guide block is rotatable relative to the wedge block by an acute angle and limited from further rotation by interference between the flattened sides of the wedge projection and the guide block opening.

16. A method of reducing an angular deformity in a joint, the method comprising:
configuring an orthopedic surgical system into a first configuration with a handle mounted to a serrated wedge to form a joint-repositioning tool;
inserting the wedge of the joint-repositioning tool between the two adjacent bones of the joint to reposition at least one of the bones and to secure the wedge in position with the serrations dug into the joint cartilage and/or bone;
reconfiguring the system into a second configuration, while the wedge remains secured in the joint, by removing the handle from the wedge and mounting a cutting guide with guide slots to the wedge to form a saw-guide tool;
inserting an orthopedic bone saw blade through at least one of the guide slots of the cutting guide to remove at least one bone end portion, without the need for any additional securement to the two adjacent bones.

17. The method of claim 16, wherein inserting the wedge includes inserting the wedge of the joint-repositioning tool between the two adjacent bones of the joint, by a selected distance into the joint, to reposition at least one of the bones at an angle corresponding to the depth of the wedge insertion, and to secure the wedge in position with the serrations dug into the joint cartilage, wherein the same wedge can be used to reposition the bone at different angles.

18. The method of claim 17, wherein inserting the wedge further includes repositioning the bones closer to but not in alignment with each other, and further comprising, before inserting an orthopedic bone saw blade, rotating the cutting guide relative to the wedge before securing the cutting guide in a fixed position relative to the wedge, or selecting an angled guide slot for the orthopedic bone saw blade to be inserted into.

19. The method of claim 17, wherein inserting the wedge further includes inserting a truncated wedge to reposition the bones in two planes.

20. The method of claim 17, further comprising:
reconfiguring the system back into the first configuration, while the wedge remains secured in the joint, by removing the cutting guide from the wedge and remounting the handle to the wedge; and
removing the wedge from the joint by applying a retraction force on the handle, wherein removing the wedge from the joint also removes the at least one bone end portion from the joint due to the serrations of the wedge being dug into the joint cartilage and/or bone.
